SEQUENCE LISTING (6 sequences)

SEQ ID NO: 1 - DNA, 812 bp, Plantago turrifera
tccggtgaagtgttcggatcgtggcgacgtgggcggttcgctgcccgcgacgtcgcgagaagttgaaccttatcatttagaggaaggagaagtcgtaacaaggtttccgtaggtgaacctgcggaaggatcattgtcgatatccaaaaagtagacctgtgaacacgtgtttaacatgaacggtgcctcgtcgggttggagcaatccactcttcgggacaccgtgcctgcccggtgcttgcacttggtgggctaacgaaacccggcgcggcaagcgccaaggaaaacaaaatggaaacgttgctccccttgactcccgttcgcggtgttgttttggggatgtgatgtatcttgaaagtcataacgactctcggcaacggatatctcggctctcgcatcgatgaagaacgtagcgaaatgcgatacttggtgtgaattgcagaatcccgtgaaccatcgagtctttgaacgcaagttgcgcccgacgccttcgggctgagggcacgcctgcctgggcgtcacgcatcgcgtcgccccctataattcggtatgggggcggataatggcatcccgttagctcggtttgcccaaaaaggatccctcatcgacggatgtcacaaccagtggtggttgaaagatcattggtgccgttgtgcttcactccgtcgcatgctcgggcatcgttataaaacaatggtgctaatgcgccttcgaccgcgaccccaggtcagacgggattacccgctgagtttaagcatatcaataagcggtggagaagaaacttacaaggattcccctagtaacggcgagcgac

SEQ ID NO: 2 - DNA, 811 bp, Plantago ovata
tccggtgaagtgttcggatcgcggcgacgtgggcggttcgctgcccgcgacgtcgcgagaagttgaaccttatcatttagaggaaggagaagtcgtaacaaggtttccgtaggtgaacctgcggaaggatcattgtcgatatctgaaaagtagacctgtgaacacgtgtttaacatgaacggtgccttgttgggccagagacatctgcttgacgaggcgccgtgcctgcttggtgctagcaccttgtgggctaacgaaacccggcgcggtaagcgtcaaggaaaacaaattagaagcgttgcccttgcagctcccgttcgcggtgtggttgtggggatgcagcgtatcttgaaagtcaaaacgactctcggcaacggatatcttggttctcgcatcgatgaagaacgtagcgaaatgcgatacttggtgtgaattgcagaatcccgtgaaccatcgagtctttgaacgcaagttgcgcccgacgccttcgggctgagggcacgcctgcctgggcgtcacgcatcgcgtcgccccctccggttcggtgatggggcggacaatggcttcccgttagctcggttagcctaaaaaggatccctcaacgatggatgtcacaaccagtggtggttgaaagatcattggtgctgttgtgcttcaccctgtcgcttgctagggcatcatcataaaccaacggcgtgaatgcgccttcgaccgcgaccccaggtcagacgggactacccgctgagtttaagcatatcaataagcggtggagaagaaacttacaaggattcccctagtaacggcgagcgac

SEQ ID NO: 3 - DNA, 812 bp, Unknown; identical to SEQ ID NO: 1
  LOX cultivar of Plantago species

SEQ ID NO: 4 - DNA, 812 bp, Unknown
  HAP cultivar of Plantago species
tccggtgaagtgttcggatcgcggcgacgtgggcggttcgctgcccgcgacgtcgcgagaagttgaaccttatcatttagaggaaggagaagtcgtaacaaggtttccgtaggtgaacctgcggaaggatcattgtcgatatccaaaaagtagacctgtgaacacgtgtttaacatgaacggtgcctcgtcgggttggagcaatccactcttcgggacaccgtgcctgcccggtgcttgcacttggtgggctaacgaaacccggcgcggcaagcgccaaggaaaacaaaatggaaacgttgctccccttgactcccgttcgcggtgttgttttggggatgtgatgtatcttgaaagtcataacgactctcggcaacggatatctcggctctcgcatcgatgaagaacgtagcgaaatgcgatacttggtgtgaattgcagaatcccgtgaaccatcgagtctttgaacgcaagttgcgcccgacgccttcgggctgagggcacgcctgcctgggcgtcacgcatcgcgtcgccccctataattcggtatgggggcggataatggcatcccgttagctcggtttgcccaaaaaggatccctcatcgacggatgtcacaaccagtggtggttgaaagatcattggtgccgttgtgcttcactccgtcgcatgctcgggcatcgttataaaacaatggtgctaatgcgccttcgaccgcgaccccaggtcagacgggattacccgctgagtttaagcatatcaataagcggtggagaagaaacttacaaggattcccctagtaacggcgagcgaa

SEQ ID NO: 5 - DNA, 28 bp, Artificial Sequence
  Forward ITS Primer
acgaattcatggtccggtgaagtgttcg

SEQ ID NO: 6 - DNA, 28 bp, Artificial Sequence
  Reverse ITS Primer
tagaattccccggttcgctcgccgttac

(19) United States
(12) Patent Application Publication
(10) Pub. No.: US 2024/0276991 A1
(43) Pub. Date: Aug. 22, 2024

(54) FOOD COMPOSITION

(71) Applicant: THE UNIVERSITY OF ADELAIDE, Adelaide, South Australia (AU)

(72) Inventors: James Morgan COWLEY, Lower Mitcham (AU); Rachel Anita BURTON, Panorama (AU)

(21) Appl. No.: 18/568,582

(22) PCT Filed: Jun. 7, 2022
(86) PCT No.: PCT/AU2022/050558

§ 371 Date: Dec. 8, 2023

(30) Foreign Application Priority Data
Jun. 8, 2021 (AU) 2021901707

(51) Int. Cl. A21D 13/047; A21D 2/14; A21D 2/18; A21D 2/38; A21D 10/00; A21D 13/066; A21D 13/068

(52) U.S. Cl. CPC A21D 13/047; A21D 2/145; A21D 2/186; A21D 2/38; A21D 10/005; A21D 13/066; A21D 13/068

(57) ABSTRACT

A food additive composition comprising flour or meal made from seeds of a plant of the genus Plantago, wherein the ω-3 to ω-6 fatty acid molar ratio of the seeds is at least 2.0:1.

### FIELD OF THE INVENTION

The present invention relates generally to a functional food additive composition with a high ω-3 to ω-6 mole ratio that can be used as a food supplement in various food applications. The invention also relates to a method of making a food additive composition of the invention, baking compositions containing the food additive compositions and methods of making baked products, especially gluten free baked products, using the food additive composition.

### BACKGROUND OF THE INVENTION

The concept of the use of functional foods to provide health benefits was developed in Japan and has since spread to the rest of the world. Health authorities in Japan in the 1980's had to deal with the prospect of an aging population and increasing life expectancy leading to elevated health care costs. They realised that the only way to contain these costs was by improvements to quality of life such that health care costs could be controlled. One way of achieving this was to promote the concept of foods to the wider population that could either promote health or reduce the risk of disease occurring. The term “functional food” was coined to describe foods of this type and the growth in research and sales of these foods has been remarkable.

Accordingly, functional foods may be defined as foods that have a positive effect on human health beyond the basic nutrition provided by the food. Functional foods are typically separated into two categories namely conventional and modified functional foods. Conventional functional foods are natural whole food ingredients that are rich in one or more important nutrients such as vitamins, minerals, probiotics, fibre and heart healthy fats. Modified functional foods that have been fortified with additional ingredients such as vitamins, minerals fibre and the like to increase the health benefit of a food.

An example of a functional food of this type are psyllium husks which are manufactured from the milled seed husk of Plantago ovata. The milled seed husk of P. ovata, contains a highly-hydrophilic hemicellulose called heteroxylan which is used to texturally mimic fat and gluten and as a dietary fibre supplement to treat defecation problems, hypercholesterolemia, diabetes, and irritable bowel syndrome. Whilst this product has many uses, P. ovata, is plagued by many agronomy-related quality issues and is wasteful as the non-mucilage-producing tissues are discarded. Accordingly, the wastage in the production of psyllium husk from P. ovata is significant as it is only the outer layer of the seed that is utilised in the production of the commercial product.

There has therefore been an ongoing search for different functional foods that can provide various health benefits to consumers and in many instances to address food imbalances that have developed in the modern world and have led to health problems. One such imbalance is the mole ratio of ω-3 to ω-6 fatty acids in the diet which is thought to have only really occurred in the last 100 years or so.

There are various sources that strongly suggest that humans typically consumed a diet that provided an ω-6 to ω-3 molar ratio of approximately 1:1 which is in stark contrast to the modern western diet where it is estimated that the molar ratio is closer to 15:1 to 17:1. This has occurred as modern western diets contain excessive amounts of ω-6 fatty acids and a correspondingly low level of ω-3 fatty acids. Whilst there are many theories as to why this has occurred, it is generally thought that the combined effect of the growth in processed foods due to modern food technology and modern agriculture has led to an increase in the consumption of ω-6 fatty acids in the diet. For example, it is thought that these changes led to the mass production of vegetable oils high in ω-6 fatty acids and changed animal feeds from the traditional grass to grains (such as in feedlot production). This is thought to have dramatically increased consumption of linoleic acid in the form of vegetable oils incorporated into processed food and arachidonic acid from meat and eggs and accordingly significantly increased the level of ω-6 fatty acids in the human diet.

As the human genome developed with a dietary molar ratio of ω-6 to ω-3 of approximately 1:1, the consumption of a diet in which this ratio is significantly skewed has the potential to cause a number of health issues in the consumer. For example, it has been found that the excessive molar ratio of ω-6 to ω-3 promotes the pathogenesis of many diseases including cancer, cardiovascular diseases and inflammatory and autoimmune diseases. In addition, consumption of a diet with a high molar ratio of ω-6 to ω-3 leads to an increase in white adipose tissue which is associated with obesity. In contrast ω-3 fatty acids typically decrease adipose tissue development and lead to weight loss.

It has also been established ω-6 and ω-3 fatty acids compete for the same conversion enzymes in the body. As a result, the amount of ω-6 fatty acids in the diet directly affects the conversion of ω-3 fatty acids such as ALA (alpha-linoleic acid) to long chain ω-3 EPA (eicosapentanoic acid) and DHA (docosahexanoic acid) which provide the body with a protective effect from diseases. In general, ω-6 fatty acids are pro-inflammatory whereas ω-3 fatty acids are neutral. Accordingly, a high ω-6 to ω-3 molar ratio in the diet increases inflammation in the body leading to an increase in the risk of inflammatory disease occurring. The list of diseases associated with inflammation include but are not limited to cardiovascular disease, type 2 diabetes, obesity, metabolic syndrome, irritable bowel syndrome and inflammatory bowel disease, macular degeneration, rheumatoid arthritis, asthma, cancer, psychiatric disorders and autoimmune diseases.

Accordingly it is well established that the currently high molar ratio of ω-6 to ω-3 in the diet is undesirable leading to an increased risk of the development of inflammatory related disorders as discussed above. There is therefore a need to provide food additives with ω-3 to ω-6 molar ratios greater than 1.0 that can be used as both food supplements and additives to improve human health. It would be expected that foods of this type would be useful in a wide variety of applications as functional foods.

There is also a growing body of evidence that ω-3 fatty acids, particularly long-chain fatty acids (LCFAs) EPA and DHA commonly obtained from consuming fish, are protective against cardiovascular diseases. However, many researchers conclude that the world's ecosystems could not sustainably supply the population with enough fish to provide the recommended EPA and DHA intake and thus sufficient intake of plant-derived UFAs to allow endogenous LCFA production from these precursors is recommended as an alternative. As plant-derived ω-3 (anti-inflammatory) and ω-6 (pro-inflammatory) UFAs are competitively desaturated and elongated into LCFAs by the same pathways, it is important that the molar ratio of ω-3 to ω-6 is at the very least 1:1 to reduce the risk of inflammation. However, an ω-3 to ω-6 intake molar ratio of 4:1 is suggested to be ideal for promoting heart, liver and gut health.

Accordingly it has been appreciated that foods containing the appropriate ratio of ω-3 to ω-6 is may form part of a balanced diet. In response to this functional food additives or supplements have been developed whereby foods have been fortified with the addition of desirable components such as ω-3 fatty acids. Whilst these compositions can provide the desired functionality they are typically expensive to manufacture due to the additional manufacturing steps required in adding the desirable component but are also seen as being less desirable as they are perceived by consumers as not being “natural” or “organic”.

The discussion of documents, acts, materials, devices, articles and the like is included in this specification solely for the purpose of providing a context for the present invention. It is not suggested or represented that any or all of these matters formed part of the prior art base or were common general knowledge in the field relevant to the present invention as it existed before the priority date of each claim of this application.

### SUMMARY OF THE INVENTION

The present invention has arisen from the desire to identify foods that can provide ω-3 to ω-6 fatty acid molar ratios greater than 1.0:1 that can be used as functional foods either alone or as food supplements or in food additive compositions. In conducting research into potential sources of functional foods of this type attention was turned towards seeds that produced mucilage as it was felt that these could provide useful properties. It was noted that upon wetting, seeds of Plantago species produce viscous polysaccharides called mucilage that has value as food additives and bulking dietary fibre. However, the only commercially-relevant species, P. ovata, is plagued by many agronomy-related quality issues and its use is wasteful as the non-mucilage-producing tissues are discarded. As a result of their studies the present applicants have identified that the seeds of certain Plantago species contain molar ratios of ω-3 to ω-6 fatty acids greater than 1.0:1. Accordingly, in a first aspect the present invention provides a food additive composition comprising flour or meal made from seeds of a plant of the genus Plantago, wherein the ω-3 to ω-6 fatty acid molar ratio of the seeds is at least 2.0:1. In one aspect the plant of the genus Plantago is Plantago turrifera or a variant or mutant thereof.

The present applicants have found that the food additive composition of the invention is particularly useful as a supplement in baking compositions as it is typically gluten free and imparts useful properties on baking compositions and the final baked products made therefrom. In a second aspect the present invention provides a baking composition comprising (1) a food additive composition of the invention and (2) flour. The baking composition may contain the food additive composition of the invention and the flour in a wide variety of ratios and may also contain other suitable baking additives well known in the art.

As discussed above, the food additive composition is particularly useful in the preparation of baking compositions and can therefore be used in method of making baked products. In yet an even further aspect the present invention provides a method of making a baked product the method comprising the steps of: (a) forming a dough comprising (1) a food additive composition according to the invention and (2) a flour; and (b) baking the dough to form a baked product. The applicants of the present invention have also found that the food additive composition can be used as a specialty baking product as the food additive of the present invention is gluten free with the result that it can be used in the preparation of gluten free baked products.

In yet a further aspect there is provided a method of making a food additive composition the method comprising providing seeds of a plant of the genus Plantago, wherein the seeds have ω-3 to ω-6 fatty acid molar ratio of at least 2.0:1, and grinding the seeds to produce a flour or meal. In one embodiment the method comprises grinding whole seeds.

### BRIEF DESCRIPTION OF THE FIGURES

For a further understanding of the aspects and advantages of the present invention, reference should be made to the following detailed description, taken in conjunction with the accompanying figures which illustrate certain embodiments of the present invention.

FIG. 1—a Monosaccharide analysis of total extracted seed mucilage indicating structural differences between Plantago ovata and Plantago turrifera.

FIG. 2—Graphs showing the effect of the addition of flour derived from Plantago ovata and Plantago turrifera on pasting qualities of rice flour and rice starch.

FIG. 3—graphs showing the effect of the addition of flour derived from Plantago ovata and Plantago turrifera on viscosity of rice flour.

FIG. 4—a graph showing effect of Plantago ovata and Plantago turrifera addition on syneresis of rice flour pastes.

FIG. 5—Cross sections of gluten-free breads after Plantago ovata and Plantago turrifera addition showing crumb structure appearance (top) and measured porosity (bottom).

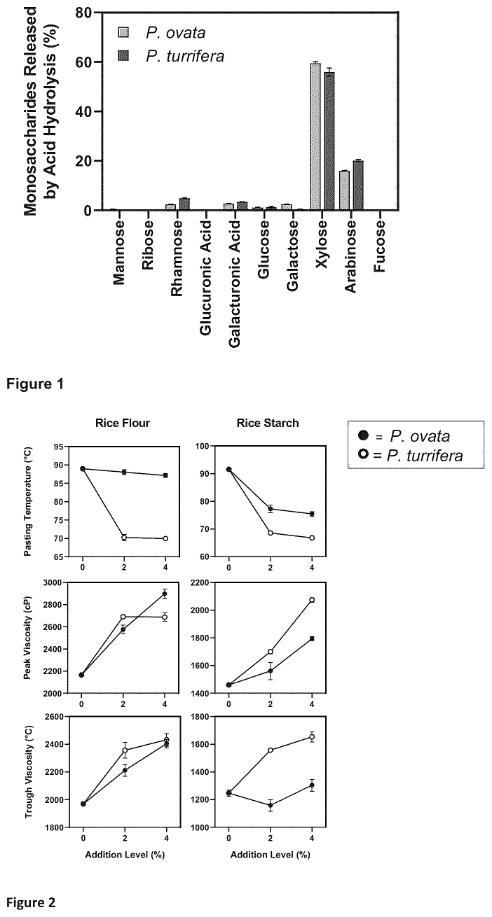

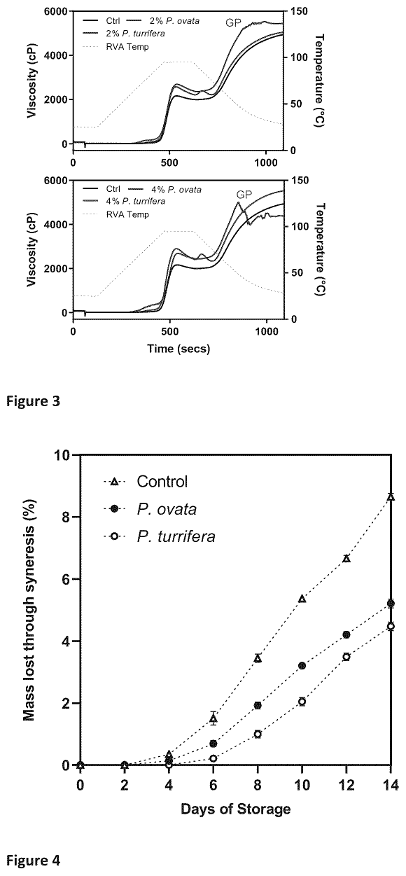

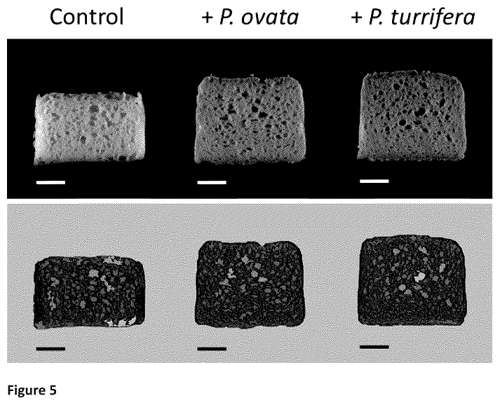

### DETAILED DESCRIPTION OF THE INVENTION

Nucleotide sequences are referred to herein by a sequence identifier number (SEQ ID NO:). A summary of the sequence identifiers is provided in Table 1. A sequence listing has also been provided at the time of filing this application.

TABLE 1
Summary of Sequence Identifiers

| Sequence Identifier | Description |
|---|---|
| SEQ ID NO: 1 | Plantago turrifera including 5.8S rRNA |
| SEQ ID NO. 2 | Plantago ovata including 5.8S rRNA |
| SEQ ID NO. 3 | Plantago [LOX Cultivar] including 5.8s rRNA |
| SEQ ID NO. 4 | Plantago [HAP cultivar] including 5.8S rRNA |
| SEQ ID NO. 5 | Forward ITS Primer |
| SEQ ID NO. 6 | Reverse ITS Primer |

It is thought that the LOX cultivar is Plantago turrifera and that the HAP cultivar is a mutant thereof.

P._turrifera
(SEQ ID NO. 1)
TCCGGTGAA GTGTTCGGAT CGTGGCGACG TGGGCGGTTC
GCTGCCCGCG ACGTCGCGAG AAGTTGAACC TTATCATTTA
GAGGAAGGAG AAGTCGTAAC AAGGTTTCCG TAGGTGAACC
TGCGGAAGGA TCATTGTCGA TATCCAAAAA GTAGACCTGT
GAACACGTGT TTAACATGAA CGGTGCCTCG TCGGGTTGGA
GCAATCCACT CTTCGGGACA CCGTGCCTGC CCGGTGCTTG
CACTTGGTGG GCTAACGAAA CCCGGCGCGG CAAGCGCCAA
GGAAAACAAA ATGGAAACGT TGCTCCCCTT GACTCCCGTT
CGCGGTGTTG TTTTGGGGAT GTGATGTATC TTGAAAGTCA
TAACGACTCT CGGCAACGGA TATCTCGGCT CTCGCATCGA
TGAAGAACGT AGCGAAATGC GATACTTGGT GTGAATTGCA
GAATCCCGTG AACCATCGAG TCTTTGAACG CAAGTTGCGC
CCGACGCCTT CGGGCTGAGG GCACGCCTGC CTGGGCGTCA
CGCATCGCGT CGCCCCCTAT AATTCGGTAT GGGGGGGGAT
AATGGCATCC CGTTAGCTCG GTTTGCCCAA AAAGGATCCC
TCATCGACGG ATGTCACAAC CAGTGGTGGT TGAAAGATCA
TTGGTGCCGT TGTGCTTCAC TCCGTCGCAT GCTCGGGCAT
CGTTATAAAA CAATGGTGCT AATGCGCCTT CGACCGCGAC
CCCAGGTCAG ACGGGATTAC CCGCTGAGTT TAAGCATATC
AATAAGCGGT GGAGAAGAAA CTTACAAGGA TTCCCCTAGT
AACGGCGAGC GAC
P._ovata
(SEQ ID NO. 2)
TCCGGTGAA GTGTTCGGAT CGCGGCGACG TGGGCGGTTC
GCTGCCCGCG ACGTCGCGAG AAGTTGAACC TTATCATTTA
GAGGAAGGAG AAGTCGTAAC AAGGTTTCCG TAGGTGAACC
TGCGGAAGGA TCATTGTCGA TATCTGAAAA GTAGACCTGT
GAACACGTGT TTAACATGAA CGGTGCCTTG TTGGGCCAGA
GACATCTGCT TGACGAGGCG CCGTGCCTGC TTGGTGCTAG
CACCTTGTGG GCTAACGAAA CCCGGCGCGG TAAGCGTCAA
GGAAAACAAA TTAGAAGCGT TGCCCTTGC AGCTCCCGTT
CGCGGTGTGG TTGTGGGGAT GCAGCGTATC TTGAAAGTCA
AAACGACTCT CGGCAACGGA TATCTTGGTT CTCGCATCGA
TGAAGAACGT AGCGAAATGC GATACTTGGT GTGAATTGCA
GAATCCCGTG AACCATCGAG TCTTTGAACG CAAGTTGCGC
CCGACGCCTT CGGGCTGAGG GCACGCCTGC CTGGGCGTCA
CGCATCGCGT CGCCCCCTCC GGTTCGGTGA TGGGGCGGAC
AATGGCTTCC CGTTAGCTCG GTTAGCCTAA AAAGGATCCC
TCAACGATGG ATGTCACAAC CAGTGGTGGT TGAAAGATCA
TTGGTGCTGT TGTGCTTCAC CCTGTCGCTT GCTAGGGCAT
CATCATAAAC CAACGGCGTG AATGCGCCTT CGACCGCGAC
CCCAGGTCAG ACGGGACTAC CCGCTGAGTT TAAGCATATC
AATAAGCGGT GGAGAAGAAA CTTACAAGGA TTCCCCTAGT
AACGGCGAGC GAC
LOX
(SEQ ID NO. 3)
TCCGGTGAA GTGTTCGGAT CGTGGCGACG TGGGGGGTTC
GCTGCCCGCG ACGTCGCGAG AAGTTGAACC TTATCATTTA
GAGGAAGGAG AAGTCGTAAC AAGGTTTCCG TAGGTGAACC
TGCGGAAGGA TCATTGTCGA TATCCAAAAA GTAGACCTGT
GAACACGTGT TTAACATGAA CGGTGCCTCG TCGGGTTGGA
GCAATCCACT CTTCGGGACA CCGTGCCTGC CCGGTGCTTG
CACTTGGTGG GCTAACGAAA CCCGGCGCGG CAAGCGCCAA
GGAAAACAAA ATGGAAACGT TGCTCCCCTT GACTCCCGTT
CGCGGTGTTG TTTTGGGGAT GTGATGTATC TTGAAAGTCA
TAACGACTCT CGGCAACGGA TATCTCGGCT CTCGCATCGA
TGAAGAACGT AGCGAAATGC GATACTTGGT GTGAATTGCA
GAATCCCGTG AACCATCGAG TCTTTGAACG CAAGTTGCGC
CCGACGCCTT CGGGCTGAGG GCACGCCTGC CTGGGCGTCA
CGCATCGCGT CGCCCCCTAT AATTCGGTAT GGGGGGGGAT
AATGGCATCC CGTTAGCTCG GTTTGCCCAA AAAGGATCCC
TCATCGACGG ATGTCACAAC CAGTGGTGGT TGAAAGATCA
TTGGTGCCGT TGTGCTTCAC TCCGTCGCAT GCTCGGGCAT
CGTTATAAAA CAATGGTGCT AATGCGCCTT CGACCGCGAC
CCCAGGTCAG ACGGGATTAC CCGCTGAGTT TAAGCATATC
AATAAGCGGT GGAGAAGAAA CTTACAAGGA TTCCCCTAGT
AACGGCGAGC GAC
HAP
(SEQ ID NO. 4)
TCCGGTGAA GTGTTCGGAT CGCGGCGACG TGGGGGGTTC
GCTGCCCGCG ACGTCGCGAG AAGTTGAACC TTATCATTTA
GAGGAAGGAG AAGTCGTAAC AAGGTTTCCG TAGGTGAACC
TGCGGAAGGA TCATTGTCGA TATCCAAAAA GTAGACCTGT
GAACACGTGT TTAACATGAA CGGTGCCTCG TCGGGTTGGA
GCAATCCACT CTTCGGGACA CCGTGCCTGC CCGGTGCTTG
CACTTGGTGG GCTAACGAAA CCCGGCGCGG CAAGCGCCAA
GGAAAACAAA ATGGAAACGT TGCTCCCCTT GACTCCCGTT
CGCGGTGTTG TTTTGGGGAT GTGATGTATC TTGAAAGTCA
TAACGACTCT CGGCAACGGA TATCTCGGCT CTCGCATCGA
TGAAGAACGT AGCGAAATGC GATACTTGGT GTGAATTGCA
GAATCCCGTG AACCATCGAG TCTTTGAACG CAAGTTGCGC
CCGACGCCTT CGGGCTGAGG GCACGCCTGC CTGGGCGTCA
CGCATCGCGT CGCCCCCTAT AATTCGGTAT GGGGGGGGAT
AATGGCATCC CGTTAGCTCG GTTTGCCCAA AAAGGATCCC
TCATCGACGG ATGTCACAAC CAGTGGTGGT TGAAAGATCA
TTGGTGCCGT TGTGCTTCAC TCCGTCGCAT GCTCGGGCAT
CGTTATAAAA CAATGGTGCT AATGCGCCTT CGACCGCGAC
CCCAGGTCAG ACGGGATTAC CCGCTGAGTT TAAGCATATC
AATAAGCGGT GGAGAAGAAA CTTACAAGGA TTCCCCTAGT
AACGGCGAGC GAA
Forward ITS Primer
(SEQ ID NO. 5)
ACGAATTCATGGTCCGGTGAAGTGTTCG
Reverse ITS Primer
(SEQ ID NO. 6)
TAGAATTCCCCGGTTCGCTCGCCGTTAC

As set out above, the present invention is predicated, in part, on the finding that the seeds of certain Plantago species contain molar ratios of ω-3 to ω-6 fatty acids greater than 1.0:1. Accordingly, in a first aspect the present invention provides a food additive composition comprising flour or meal made from seeds of a plant of the genus Plantago, wherein the ω-3 to ω-6 fatty acid molar ratio of the seeds is at least 2.0:1.

In yet an even further aspect the invention provides a method of making a food additive composition, the method comprising providing seeds of a plant of the genus Plantago, wherein the seeds have ω-3 to ω-6 fatty acid molar ratio of at least 2.0:1, and grinding the seeds to produce a flour or meal. In one embodiment the method comprises grinding whole seeds.

The grinding of the whole seeds of the plant of the genus Plantago may be carried out in any way known in the art. This typically involves either the use of conventional milling such as roller milling although in principle any grinding technique may be used.

The applicants have therefore identified that the seeds of certain plants of the genus Plantago provide molar ratios of ω-3 to ω-6 fatty acids that are greater than 1.0:1 and which may be used in food additive compositions. In particular, the applicants have found that plants from the genus Plantago that contain a sequence ID No 1 or contain a sequence with appropriate sequence identity to sequence ID No 1 will provide seeds with the desired ω-3 to ω-6 fatty acid molar ratios.

In certain embodiments the plants of the genus Plantago contain a sequence having at least 95% sequence identity to sequence ID No 1. In certain embodiments the plants of the genus Plantago contain a sequence having at least 95.5% sequence identity to sequence ID No 1. In certain embodiments the plants of the genus Plantago contain a sequence having at least 96% sequence identity to sequence ID No 1. In certain embodiments the plants of the genus Plantago contain a sequence having at least 96.5% sequence identity to sequence ID No 1. In certain embodiments the plants of the genus Plantago contain a sequence having at least 97% sequence identity to sequence ID No 1. In certain embodiments the plants of the genus Plantago contain a sequence having at least 97.5% sequence identity to sequence ID No 1. In certain embodiments the plants of the genus Plantago contain a sequence having at least 98% sequence identity to sequence ID No 1. In certain embodiments the plants of the genus Plantago contain a sequence having at least 98.5% sequence identity to sequence ID No1. In certain embodiments the plant of the genus Plantago is a species that contains a sequence having at least 99% sequence identity to sequence ID No 1. In certain embodiments the plant of the genus Plantago is a species that contains a sequence having at least 99.5% sequence identity to sequence ID No 1.

As would be appreciated by a person of skill in the art certain plants of the genus Plantago have higher sequence identity to sequence Id No 1 than others. In one embodiment the plant of the genus Plantago is Plantago turrifera or a variant or mutant thereof. In one specific embodiment the plant of the genus Plantago is Plantago turrifera.

In general the food additive composition will contain flour or meal made from whole seeds of a single plant of the genus Plantago as this ensures that the ω-3 to ω-6 fatty acid molar ratio can be most reliably controlled as the applicants have found that whilst there is variation in the fatty acid molar ratio between species the variation within seeds of the same species is immaterial. Accordingly, in one embodiment the food additive composition comprises flour or meal made from a single species of the genus Plantago.

In other embodiments the food additive composition comprises flour or meal made from whole seeds of several different species of plants from the genus Plantago. In one embodiment the food additive composition comprises flour or meal derived from two species of plants from the genus Plantago. In one embodiment the food additive composition comprises flour or meal derived from three species of plants from the genus Plantago. In one embodiment the food additive composition comprises flour or meal derived from four species of plants from the genus Plantago. In one embodiment the food additive composition comprises flour or meal derived from five species of plants from the genus Plantago.

As discussed above the food additive composition of the present invention comprises flour or meal made from seeds of a plant of the genus Plantago wherein the seeds have an ω-3 to ω-6 fatty acid molar ratio of at least 2.0:1. In one embodiment the seeds have an ω-3 to ω-6 fatty acid molar ratio of at least 3.0:1. In one embodiment the seeds have an ω-3 to ω-6 fatty acid molar ratio of at least 4.0:1. In one embodiment the seeds have an ω-3 to ω-6 fatty acid molar ratio of at least 4.1:1. In one embodiment the seeds have an ω-3 to ω-6 fatty acid molar ratio of at least 4.2:1. In one embodiment the seeds have an ω-3 to ω-6 fatty acid molar ratio of at least 4.3:1. In one embodiment the seeds have an ω-3 to ω-6 fatty acid molar ratio of at least 4.4:1. In one embodiment the seeds have an ω-3 to ω-6 fatty acid molar ratio of at least 4.5:1. In one embodiment the seeds have an ω-3 to ω-6 fatty acid molar ratio of at least 4.6:1. In one embodiment the seeds have an ω-3 to ω-6 fatty acid molar ratio of at least 4.7:1. In one embodiment the seeds have an ω-3 to ω-6 fatty acid molar ratio of at least 4.8:1.

In certain embodiments the amount of flour or meal in the food additive composition is such that the food additive composition itself has an ω-3 to ω-6 fatty acid molar ratio of at least 2.0:1. In one embodiment the food additive composition of the present invention has an ω-3 to ω-6 fatty acid molar ratio of at least 3.0:1. In one embodiment the food additive composition of the present invention has an ω-3 to ω-6 fatty acid molar ratio of at least 4.0:1. In one embodiment the food additive composition of the present invention has an ω-3 to ω-6 fatty acid molar ratio of at least 4.1:1. In one embodiment the food additive composition of the present invention has an ω-3 to ω-6 fatty acid molar ratio of at least 4.2:1. In one embodiment the food additive composition of the present invention has an ω-3 to ω-6 fatty acid molar ratio of at least 4.3:1. In one embodiment the food additive composition of the present invention has an ω-3 to ω-6 fatty acid molar ratio of at least 4.4:1. In one embodiment the food additive composition of the present invention has an ω-3 to ω-6 fatty acid molar ratio of at least 4.5:1. In one embodiment the food additive composition of the present invention has an ω-3 to ω-6 fatty acid molar ratio of at least 4.6:1. In one embodiment the food additive composition of the present invention has an ω-3 to ω-6 fatty acid molar ratio of at least 4.7:1. In one embodiment the food additive composition of the present invention has an ω-3 to ω-6 fatty acid molar ratio of at least 4.8:1.

The food additive composition typically contains a number of fatty acids including both saturated fatty acids and unsaturated fatty acids. Saturated fatty acids contained in the composition may include myristic acid, pentadecylic acid, palmitic acid, margaric acid, stearic acid, arachidic acid, behenic acid and lignoceric acid. Unsaturated fatty acids contained in the food additive composition include alpha-linolenic acid, linoleic acid, eicosadienoic acid, palmitoleic acid, cis-vaccenic acid, oleic acid and gondoic acid.

A particularly relevant fatty acid contained in the food additive composition of the present invention is alpha linoleic acid. In one embodiment the food additive composition of the invention contains alpha linoleic acid in an amount of at least 5.0 wt % based on the total weight of the composition. In one embodiment the food additive composition of the invention contains alpha linoleic acid in an amount of at least 5.1 wt % based on the total weight of the composition. In one embodiment the food additive composition of the invention contains alpha linoleic acid in an amount of at least 5.2 wt % based on the total weight of the composition. In one embodiment the food additive composition of the invention contains alpha linoleic acid in an amount of at least 5.3 wt % based on the total weight of the composition. In one embodiment the food additive composition of the invention contains alpha linoleic acid in an amount of at least 5.4 wt % based on the total weight of the composition. In one embodiment the food additive composition of the invention contains alpha linoleic acid in an amount of at least 5.5 wt % based on the total weight of the composition. In one embodiment the food additive composition of the invention contains alpha linoleic acid in an amount of at least 5.6 wt % based on the total weight of the composition. In one embodiment the food additive composition of the invention contains alpha linoleic acid in an amount of at least 5.7 wt % based on the total weight of the composition. In one embodiment the food additive composition of the invention contains alpha linoleic acid in an amount of at least 5.8 wt % based on the total weight of the composition. In one embodiment the food additive composition of the invention contains alpha linoleic acid in an amount of at least 5.9 wt % based on the total weight of the composition. In one embodiment the food additive composition of the invention contains alpha linoleic acid in an amount of at least 6.0 wt % based on the total weight of the composition. In one embodiment the food additive composition of the invention contains alpha linoleic acid in an amount of at least 6.1 wt % based on the total weight of the composition. In one embodiment the food additive composition of the invention contains alpha linoleic acid in an amount of at least 6.2 wt % based on the total weight of the composition

A further feature of the food additive composition of the invention is its ability to provide gelling properties to other food compositions it is added to. Without wishing to be bound by theory it is felt that this ability is derived from the mucilage of the seed of the species of Plantago that the flour or meal is derived from. Mucilage is a gelatinous substance secreted by plants composed of proteins and polysaccharides and is found in a number of seeds. Whilst there are a number of proposed reasons as to why seeds produce mucilage it is thought, due to its speed of formation, that its purpose is to retain moisture around the seed. In this way it is thought that the gel could regulate the amount of moisture that reaches the seed and growing embryo. It is also thought that the polysaccharides will put water into the seed when required as the seed dries and will repel water once it reaches saturation point. Accordingly, the mucilage serves an important purpose in protection of the seed.

The applicants have found that certain mucilage compositions provide the food composition with improved properties especially the ability of the food additive composition to act as a gelling or stiffening agent. In Plantago species heteroxylan is the main constituent of the mucilage. Xylose comprises the heteroxylan backbone and, as such, the ratio of arabinose to xylose (A:X) components can be used as an estimation of backbone branching. In one embodiment the molar ratio of arabinose to xylose in the mucilage of the seed of the plant of the Plantago species is at least 0.3:1. In one embodiment the molar ratio of arabinose to xylose in the mucilage of the seed of the plant of the Plantago species is at least 0.31:1. In one embodiment the molar ratio of arabinose to xylose in the mucilage of the seed of the plant of the Plantago species is at least 0.32:1. In one embodiment the molar ratio of arabinose to xylose in the mucilage of the seed of the plant of the Plantago species is at least 0.33:1 In one embodiment the molar ratio of arabinose to xylose in the mucilage of the seed of the plant of the Plantago species is at least 0.34:1. In one embodiment the molar ratio of arabinose to xylose in the mucilage of the seed of the plant of the Plantago species is at least 0.35:1. In one embodiment the molar ratio of arabinose to xylose in the mucilage of the seed of the plant of the Plantago species is at least 0.36:1.

The food additive composition of the present invention is provided in the form of a flour or a meal. As used herein the term “flour” refers to a powder material made from grinding of a seed. As would be appreciated by a skilled worker in the art the flour particles can be of widely varying sizes depending upon the nature of the grind required by the end use application. Nevertheless, the flour particles typically have a weight average particle size of from 1 to 1000 microns. In one embodiment the particles range from 1 to 500 microns. In one embodiment the particles range from 10 to 500 microns. In one embodiment the particles range from 10 to 100 microns. In one embodiment the particles range from 20 to 100 microns. In one embodiment the particles range from 30 to 100 microns. In one embodiment the particles range from 40 to 100 microns. In one embodiment the particles range from 50 to 100 microns. As would be appreciated “meal” as used herein refers to a coarser grind such that the seed has been reduced to a particulate which is coarser than a flour.

The food additive composition of the present invention may contain a number of additional ingredients depending upon the desired end use application. For example, the composition may contain one or more bleaching agents, maturing agents, baking modifiers and preservatives. Examples of suitable additives include Potassium bromate, benzoyl peroxide, ascorbic acid, chlorine gas, chlorine dioxide, calcium peroxide, azodicarbonamide, calcium propanoate, sodium benzoate, tricalcium phosphate and butylated hydroxyanisole. The amount and type of additive that may be added will depend upon the desired end use application of the food additive composition and can readily be varied by the skilled worker in the field. Nevertheless, the total of all additives is typically from 1 wt % to 5 wt %.

As discussed above the food additive composition of the present invention can be used as a supplement in a number of different foods in order to provide the consumer with a more balanced fatty acid ratio in their final diet. In principle the food additive composition may be added into almost any foods such as beverages, casseroles and the like. Indeed, in principle the food additive composition could be added to any food to be consumed.

It is noted, however, that it is preferable to incorporate the food additive composition into a food such that the consumer does not appreciate that they are consuming a food containing an additive. One way of incorporating the food additive composition is in a baked product. Baked products such as bread constitute a significant portion of many western diets and are therefore seen as very attractive carriers for a functional food such as the food additive composition of the present invention.

The food additive composition of the present invention is gluten free and is very useful in the preparation of gluten free food products especially baked products. This is of significant commercial value due to the demand for gluten free products in society. Coeliac disease is a chronic autoimmune enteropathy triggered by ingestion of gluten proteins from wheat, barley and rye. Gluten-related immune responses in the digestive system trigger gastrointestinal irritation and inflammatory lesions, eventually leading to villi atrophy and associated malabsorption conditions. Currently the only effective treatment for the 1% to 2% of the population that suffer from coeliac disease and other gluten-related disorders is adherence to a strict, lifelong gluten-free (GF) diet. Accordingly, the market for gluten free products is ever-growing.

Leavened breads are arguably the most difficult baked goods to create without gluten. In breadmaking, the entanglement of gluten proteins creates a mechanically-robust three-dimensional network capable of trapping CO₂ during leavening allowing a dough to rise effectively giving it an airy yet robust texture, essential to consumers. GF breads are unable to rise effectively and so have low volume, have a powdery or crumbly texture and high crumb hardness. GF bread must therefore be augmented with ingredients and additives that can replicate the viscoelastic properties of gluten in order to produce products with satisfactory consumer appeal. Non-gluten flours and purified starches are the most common base ingredient which is often combined with one or more hydrocolloids to strengthen and viscosify the dough. The most commonly-used polymer for gluten replacement is hydroxypropylmethylcellulose (HPMC), a semi-synthetic derivative of cellulose with excellent viscoelastic properties. While HPMC is found in 40%-50% of GF breads, consumers tend to respond negatively to such synthetic hydrocolloids in the ingredient list, making natural polysaccharides, like those from plants, a more appealing alternative.

The applicants have found that the food additive composition of the present invention provides a suitable additive for the production of baked products in general and gluten free baked products in particular.

Accordingly, in yet an even further aspect the present invention provides a baking composition comprising (a) a food additive composition of the invention and (2) flour. As will be appreciated by a worker of skill in the art the amounts of (1) food additive composition and (2) flour present in the baking composition may vary significantly.

The amount of food additive composition in the baking composition of the invention is typically from 1 wt % to 10 wt %. In one embodiment the amount of food additive composition in the baking composition is from 1 wt % to 6 wt %. In one embodiment the amount of food additive composition in the baking composition is t from 2 wt % to 6 wt %. In one embodiment the amount of food additive composition in the baking composition is from 2 wt % to 5 wt %. In one embodiment the amount of food additive composition in the baking composition is from 2 wt % to 4 wt %. In one embodiment the amount of food additive composition in the baking composition is about 1 wt %. In one embodiment the amount of food additive composition in the baking composition is about 2 wt %. In one embodiment the amount of food additive composition in the baking composition is about 3 wt %. In one embodiment the amount of food additive composition in the baking composition is about 4 wt %. %. In one embodiment the amount of food additive composition in the baking composition is about 5 wt %. %. In one embodiment the amount of food additive composition in the baking composition is about 6 wt %.

The amount of flour in the baking composition is typically from 90 wt % to 99 wt %. In one embodiment the amount of flour in the baking composition is from 94 wt % to 99 wt %. In one embodiment the amount of flour composition in the baking composition is from 94 wt % to 98 wt %. In one embodiment the amount of flour in the baking composition is from 95 wt % to 98 wt %. In one embodiment the amount of flour in the baking composition is from 96 wt % to 98 wt %. In one embodiment the amount of flour in the baking composition is about 99 wt %. In one embodiment the amount of flour in the baking composition is about 98 wt %. In one embodiment the amount of flour in the baking composition is about 97 wt %. In one embodiment the amount of flour in the baking composition is about 96 wt %. %. In one embodiment the amount of flour in the baking composition is about 95 wt %. %. In one embodiment the amount of flour in the baking composition is about 94 wt %.

In one embodiment the baking composition of the invention comprises from 1 wt % to 10 wt % of the food additive composition and from 90 wt % to 99 wt % flour. In one embodiment the baking composition of the invention typically comprises from 1 wt % to 6 wt % of the food additive composition and from 94 wt % to 99 wt % flour. In one embodiment the baking composition of the invention typically comprises from 2 wt % to 6 wt % of the food additive composition and from 94 wt % to 98 wt % flour. In one embodiment the baking composition of the invention typically comprises from 2 wt % to 5 wt % of the food additive composition and from 95 wt % to 98 wt % flour. In one embodiment the baking composition of the invention typically comprises from 2 wt % to 4 wt % of the food additive composition and from 96 wt % to 98 wt % flour. In one embodiment the baking composition of the invention typically comprises from 3 wt % to 5 wt % of the food additive composition and from 95 wt % to 97 wt % flour.

The flour used in the baking composition may be any suitable flour. Examples of suitable flours include wheat flour, barley flour, corn flour, acorn flour, almond flour, amaranth flour, apple flour, banana flour, bean flour, brown rice flour, buckwheat flour, cassava flour, chestnut flour, chickpea flour, chuno flour, coconut flour, coffee flour, pea flour, peanut flour, potato starch flour, rice flour, sorghum flour, tapioca flour and teff flour.

In one preferred embodiment the flour is a gluten free flour. In certain embodiments the gluten free flour is selected from the group consisting of almond flour, brown rice flour, buckwheat flour, chickpea flour, corn flour, rice flour, sorghum flour, tapioca flour, teff flour or a combination thereof. In one embodiment the flour is almond flour. In one embodiment the flour is brown rice flour. In one embodiment the flour is buckwheat flour. In one embodiment the flour is chickpea flour. In one embodiment the flour is corn flour. In one embodiment the flour is rice flour. In one embodiment the flour is sorghum flour. In one embodiment the flour is tapioca flour. In one embodiment the flour is teff flour.

The baking composition of the invention is used in conventional ways known in the art. It is typically converted into a dough followed by baking of the dough to form a baked product. Accordingly, the present invention further provides a method of making a baked product, the method comprising (1) forming a dough containing the baking composition of the invention and (2) baking the dough to form a baked product.

In addition to the baking composition of the present invention as described above the dough may contain one or more additional additives depending upon the desired end use application. As will be appreciated the list of potential additives is fundamentally endless depending upon the type of baked product to be produced. As will be readily understood, however, there are a number of additives that are commonly found in dough in addition to the baking composition of the present invention. Examples of common additives include yeast, liquids, sweeteners, salt, eggs and fats.

In certain embodiments the dough contains yeast. Yeast is an ingredient that is at the heart of many baking processes especially the breadmaking process. The role of yeast is to make the dough rise and gives the baked product both texture and aroma. When mixed in a dough with liquids and fed by either sugar or starches in the dough the yeast activates and releases tiny bubbles of carbon dioxide which make the dough rise leading to the formation of a baked product with a light texture after baking.

The dough will almost invariably contain one or more liquids. A number of different liquids may be used such as water, milk, buttermilk, cream or juice. Water is the most common liquid in a dough and plays the twin roles of (1) helping to dissolve and activate the yeast and (2) it blends with the baking composition to help create a sticky elastic dough. Other liquids such as milk, buttermilk, cream or juice can be used in addition to or instead of the water (as they all inherently contain certain amounts of water). These are used as modifiers to either the flavour or the texture of the final baked product.

In certain embodiments the dough contains a sweetener. Examples of suitable sweeteners include brown sugar, honey, molasses, jams and white sugar. These ingredients add both flavour and colour to the baked product.

In certain embodiments the dough contains eggs. Eggs add significant food value, colour and flavour to baked products and are used extensively. They also add richness to the final baked product and add protein. In relation to bread making the addition of eggs helps to make the crumb fine and to help keep the crust tender.

In certain embodiments the dough contains added fats. The added fat may be in the form of butter, margarine, shortening, oil or a combination thereof. They play a variety of roles in baking but are typically acknowledged to add flavour and to help keep the baked product moist and tender. The presence of fat tends to slow moisture loss thus helping the baked product to stay fresh longer.

The dough may also contain one or more additional ingredients depending upon the final end use application of the dough. For example, the dough may contain added fruit such as raspberries, blackberries, currants, raisins, dried apple, dried fig and the like. The dough may also contain herbs such as basil, rosemary and oregano; nuts such as almonds, walnuts and pine nuts, flakes such as coconut flakes, rye flake and almond flakes, seeds such as poppy seeds, pepitas and fennel seeds, spices such as cinnamon, cloves and chilli, vegetables such as garlic, pumpkin or olives and proteins such as cheese, bacon and anchovies.

Once formed with the desired ingredients the dough is then baked to form a baked product. A particularly suitable baked product is bread.

The invention is further illustrated in the following examples. The examples are for the purpose of describing particular embodiments only and are not intended to be limiting with respect to the above description.

### EXAMPLES

As a result of their studies the applicants have identified that certain Plantago species provide elevated ω-3 to ω-6 molar ratios and can therefore be used as a functional food. In the following examples the applicants demonstrate the seeds of the identified Plantago species in a comparative fashion to the only commercially relevant Plantago species, Plantago ovata, which as discussed above suffers from a number of agronomy-related quality issues and is wasteful as the non-mucilage-producing tissues are disposed.

Rice flour was purchased from Doves Farms (United Kingdom). Caster sugar, table salt and sunflower oil were purchased from Sainsbury's (UK) and yeast (Saccharomyces cerevisiae) was purchased from Allinson (UK). Seeds of Plantago species were obtained and grown to maturity to produce bulk seed. Seed was ground to flour using a MM400 Mixer Mill (Retsch, Germany) and graded to 0.5 mm. Dry ingredients and oil were stored dry at room temperature. Yeast was stored dry at +4° C. Flours were stored dry at room temperature.

### Example 1—Plant Growth

Seeds of Plantago turrifera and Plantago ovata species were obtained. Seeds were vernalised dry for 48 hours at −20° C. prior to germination. Seeds were imbibed in filtered (0.22 μm) sterilisation agent (50:50, 50% ethanol:4% bleach with 0.05% Triton X-100) for 1 min before replacing the sterilisation agent and incubating for another minute. This was repeated until the seeds had been imbibed in fresh sterilisation agent 5 times, after which the seeds were washed 5 times with filter-sterilised (0.22 μm) Milli-Q water. Seeds were spread onto pre-wetted autoclaved Whatman No. 1 paper in a sterile petri dish. Dishes were sealed, aluminium foil-wrapped and vernalised for another 48 hours at 4° C. After vernalisation, plates were moved to a glasshouse with a day/night temperature of 23° C./18° C. Seeds were germinated for 10 days (3 days dark then 7 days exposed to the glasshouse day/night light cycle) then transferred to coco-peat soil mixture in tall citrus pots. Plants were grown to maturity from June to December (Adelaide, Australia) with no supplemental light.

### Example 2—Phylogenetic Analysis

Mature leaf tissue from Plantago plants were frozen at −80° C. and ground by stainless steel ball bearing for 30 sec at 30 Hz using a MM400 Mixer Mill (Retsch, Germany) fitted with 2 ml tube adapter. DNA was extracted from ground leaf tissue following Healey et al. (2014). Nuclear ribosomal DNA (nrDNA) internal transcribed spacer (ITS) regions were amplified by PCR using primers (Sun et al., 1994) and conditions listed in the table below:

TABLE 2
PCR parameters for amplification of nuclear ribo-
somal DNA internal transcribed spacer (ITS)
regions used to produce Plantago phylogenetic
tree.
Primers

| Forward ITS Primer | ACGAATTCATGGTCCGGTGAAGTGTTCG |
|---|---|
| (5′→ 3′) | |
| Reverse ITS Primer | TAGAATTCCCCGGTTCGCTCGCCGTTAC |
| (5′→ 3′) | |

PCR Conditions

| | |
|---|---|
| Activation | 95° C. for 2 min |
| Amplification Cycles | 24 Cycles |
| Denaturation | 95° C. for 30 sec |
| Annealing & Extension | 72° C. for 1 min |
| (Two-step PCR) | |
| Final Extension | 72° C. for 5 min |

Amplified ITS regions were sequenced by AGRF (Adelaide, Australia). ITS sequences were trimmed using BMGE (Criscuolo and Gribaldo, 2010) and the neighbour-joining comparison tree constructed in Geneious v8.1.3 (Biomatters Ltd, NZ) with the RAxML tree builder (Stamatakis, 2006) using the GTR GAMMA nucleotide model with 500 rapid bootstrapping replicates.

Alignment revealed many sequence discrepancies between P. ovata reference and the P. turrifera reference along with LOX and HAP accessions. Seventy polymorphisms were found in P. ovata leading to 91.4% sequence similarity with the other three accessions. No polymorphisms were found between the P. turrifera reference and the LOX accession (100% sequence similarity) while 2 polymorphisms were identified in the HAP accession leading to 99.75% similarity with P. turrifera reference and LOX.

TABLE 3
Sequence identity cross reference

| Sequence | P. turrifera | P. ovata | LOX | HAP |
|---|---|---|---|---|
| P. turrifera | 100% | 91.379% | 100% | 99.754% |
| P. ovata | 91.379% | 100% | 91.379% | 91.379% |
| Lox | 91.379% | 91.379% | 100% | 99.754% |
| HAP | 91.379% | 91.379% | 99.754% | 100% |

### Example 3—Seed Morphometric Measurements

Seed length and width measurements were determined by image analysis. Images of seeds were taken at 1× magnification on an Axiolmager M2 (Zeiss, Germany) fitted with an AxioCam 105 color camera (Zeiss, Germany). Length and width of 20 seeds per species were measured using ZEN 2012 software (Zeiss, Germany). To determine 1000 seed weight, seeds were manually-counted and weighed. The results are shown in the following Table 4.

TABLE 4
Seed physical parameters

| Seed physical property | | |
|---|---|---|
| (average) | Plantago ovata | Plantago turrifera |
| Length (mm) | 2.843 | 1.562 |
| Width (mm) | 1.448 | 0.840 |
| 1000 grain weight (mg) | 1618 | 355 |

### Example 4—Lipid Analysis

Total lipid in whole seed flour was determined by modified Folch method (1957) and fatty acid profiles were determined by gas chromatography of transesterified lipids following Liu et al. (2014). The results are shown in table 5 below.

TABLE 5
Lipid content (molar %)

| | Plantago ovata | Plantago turrifera |
|---|---|---|
| Total Fat Content (%) | 7.08 ± 0.58 | 11.51 ± 0.81 |
| Saturated Fatty Acids (total) % | 17.20 ± 0.14 | 14.37 ± 0.67 |
| Myristic Acid (14:0) % | 0.06 ± 0.03 | 0.10 ± 0.02 |
| Pentadecylic Acid (15:0) % | 0.12 ± 0.01 | 0.05 ± 0.01 |
| Palmitic Acid (16:0) % | 12.43 ± 0.13 | 11.59 ± 0.45 |
| Margaric Acid (17:0) % | 0.11 ± 0.01 | 0.09 ± 0.02 |
| Stearic Acid (18:0) % | 3.82 ± 0.06 | 2.07 ± 0.18 |
| Arachidic Acid (20:0) % | 0.42 ± 0.00 | 0.24 ± 0.00 |
| Behenic Acid (22:0) % | 0.19 ± 0.03 | 0.12 ± 0.01 |
| Lignoceric Acid (24:0) % | 0.05 ± 0.06 | 0.10 ± 0.00 |
| Unsaturated Fatty acids (total) % | 82.76 ± 0.08 | 85.58 ± 0.59 |
| Total ω-3% | 3.18 ± 0.12 | 54.46 ± 0.71 |
| Alpha-Linoleic Acid (18:3 n-3) % | 3.18 ± 0.12 | 54.46 ± 0.71 |
| Total ω-6% | 39.72 ± 0.29 | 11.30 ± 0.16 |
| Linoleic Acid (18:2 n-6) % | 39.65 ± 0.31 | 11.26 ± 0.15 |
| Eicosadienoic Acid (20:2 n-6) % | 0.07 ± 0.02 | 0.04 ± 0.01 |
| Total ω-7% | 1.37 ± 0.03 | 1.66 ± 0.02 |
| Palmitoleic Acid (16:1 n-7) % | 0.16 ± 0.02 | 0.26 ± 0.02 |
| cis-Vaccenic Acid (18:1 n-7) % | 1.20 ± 0.05 | 1.40 ± 0.01 |
| Total ω-9% | 38.49 ± 0.47 | 18.15 ± 0.07 |
| Oleic Acid (18:1 n-9) % | 38.12 ± 0.49 | 18.07 ± 0.08 |
| Gondoic Acid (20:1 n-9) % | 0.36 ± 0.02 | 0.08 ± 0.01 |
| ω-3 to ω-6 molar ratio | 0.11 ± 0.04 | 4.85 ± 0.09 |
| yield of alpha-linoleic acid | 0.23 ± 0.01 | 6.27 ± 0.52 |
| (% w/w of whole seed) | | |

As can be seen from the table P. turrifera has a significantly higher fat content than P. ovata. The fatty acid profiles are starkly different, where P. turrifera is rich in omega-3 (ω-3) fatty acids whereas in P. ovata, omega-6 (ω-6) fatty acids dominate. The omega-3 to omega-6 molar ratio, an important nutritional indicator, is 44 times higher in P. turrifera and the total yield of ALA, the most nutritionally-important omega-3 fatty acid is 27 times higher.

### Example 5—Seed Mucilage Extraction and Analysis

Mucilage was fractionated following Cowley et al. (2020) with little deviation from the described procedure.

### Monosaccharide Analysis

Monosaccharide profiles of fractionated mucilage (redispersed at 1 mg/ml in Milli-Q water) and milled whole seed flour were determined using reverse phase high performance liquid chromatography (RP-HPLC) of 1-phenyl-3-methyl-5-pyrazoline (PMP) derivatives following Hassan et al. (2017). Area under the peaks was compared to standard curves of mannose, ribose, rhamnose, glucuronic acid, galacturonic acid, glucose, galactose, xylose, arabinose and fucose (Wood et al., 2018).

TABLE 6
Total Mucilage monosaccharide Composition

| Component | Plantago ovata | Plantago turrifera |
|---|---|---|
| D-Mannose | 0.2 ± 0.3 | nd |
| D-Rhamnose | 2.4 ± 0.1 | 4.9 ± 0.1 |
| D-Glucuronic Acid | nd | nd |
| D- Galacturonic Acid | 2.8 ± 0.1 | 3.4 ± 0.1 |
| D-Glucose | 1.1 ± 0.2 | 1.3 ± 0.4 |
| D-Galactose | 2.5 ± 0.1 | 0.5 ± 0.1 |
| D-Xylose | 59.4 ± 0.7 | 55.9 ± 1.7 |
| L-Arabinose | 16.0 ± 0.2 | 20.1 ± 0.5 |

TABLE 7
Xylan content

| | P. ovata | P. turrifera | P Value |
|---|---|---|---|
| Xylan (Xylose and | 75.43 ± 0.89 | 76.03 ± 2.19 | 0.631 |
| Arabinose) Content | | | |
| (A + X %) | | | |
| Xylose to Arabinose | 1:0.27 ± 0.0003 | 1:0.36 ± 0.0016 | 0.007*** |
| molar Ratio | | | |
| (X:A) | | | |

Heteroxylan is the main constituent of the mucilage in P. ovata and P. turrifera. Importantly, the content of heteroxylan (assumed to be the content of xylose and arabinose combined) does not differ significantly between the two species (P>0.05). However, as xylose comprises the heteroxylan backbone, the ratio of xylose to arabinose (X:A) components can be used as an estimation of backbone branching. The ratio of xylose to arabinose in Plantago turrifera mucilage is significantly different than that of Plantago ovata (P<0.01). These data indicate that Plantago turrifera mucilage heteroxylan has a greater number of backbone substitutions, meaning that it is structurally distinct from that of Plantago ovata

### Example 6—Preparation of Rice Flour/Plantago Food Additive Suspensions

Fifty milligrams (2% addition) or 100 mg (4% addition) of Plantago food additive was blended with 2.5 g of rice flour or rice starch. Twenty-four grams of deionised water was added to the mixture and homogenised briefly by vigorous stirring. Control samples lacking Plantago food additive were prepared following the same protocol.

### Example 7—Effect of P. ovata and P. Turrifera Addition on Pasting Qualities of Rice Flour and Rice Starch

Pasting properties of rice flour-Plantago food additive and rice starch-Plantago food additive blends were determined by Rapid Visco-Analysis using an RVA Super 4 (Newport Scientific, Australia) based on Diaz-Calderon et al. (2018) with modifications. Suspensions were prepared as above in the RVA canister. Samples were held at 25° C. for 2 min before heating to 95° C. at 12ºC/min, holding at 95° C. for 2.5 min before cooling to 25° C. at 12° C./min where they were held for a final 2 min. For the first minute, the suspension was homogenised at 960 RPM. The remainder of the test was performed under constant stirring at 160 RPM.

The pasting parameters extracted were: pasting temperature (the temperature at which viscosity rapidly increases as a result of starch granule swelling upon the uptake of water), peak viscosity (the maximum viscosity obtained during heating), trough viscosity (the minimum viscosity obtained during hold at maximum temperature), breakdown viscosity (difference between peak and trough viscosities), final viscosity (viscosity at completion of the run), and setback viscosity (difference between final and trough viscosities). Pasting properties were measured in triplicate.

Rapid Visco Analysis was used to observe the effect that Plantago ovata and Plantago turrifera addition has on the transformation of rice flour and rice starch during cooking.

The pasting temperature is the point at which viscosity of a flour/starch paste swells upon the rapid uptake of water. A decrease in pasting temperature upon the addition of an additive corresponds to that additive's influence on the system's water uptake. In the case of hydrocolloids like heteroxylan, it corresponds to water affinity. The 2% and 4% additions of P. ovata did not significantly change the pasting temperature of rice flour (P>0.05) while P. turrifera had a highly significant effect (P=2×10⁻⁷). In rice starch, a more pure, easily-influenced system, P. ovata did significantly reduce the pasting temperature, but the effect of P. turrifera was over 65% greater.

Peak viscosity refers to the maximum viscosity of swollen flour/starch particles. An additional gel matrix can reinforce the swollen particles and increase the peak viscosity. Effects in rice flour were similar between P. ovata and P. turrifera but P. turrifera increased the peak viscosity of rice starch two-fold at both 2% and 4% addition levels.

Trough viscosity—the viscosity of the system when held at heat-refers to the stability of the system at heat and resistance to paste breakdown. Hydrocolloids like heteroxylan influence this by retaining water and maintaining a gel-like scaffold in the paste. While the effects between P. ovata and P. turrifera were similar in rice flour, in rice starch (a more pure system) the protective effect of P. turrifera compared to P. ovata is significant. The gel-like scaffold theory is supported also in the pasting profiles containing P. turrifera where the gelling point was reached. This does not occur in P. ovata-containing formulas.

Tables 8 and 9 show the pasting parameters at 2 wt % and 4 wt % of the food additive composition of the invention to rice flour.

TABLE 8
2% sample addition

| Pasting | | | |
|---|---|---|---|
| Parameter | Control | P. ovata | P. turrifera |
| Pasting | 88.95 ± 0.31^{b} | 88.03 ± 0.69^{b} | 70.25 ± 0.87^{a} |
| Temperature (° C.) | | | |
| Peak Viscosity | 2166 ± 6^{a} | 2576 ± 40^{b} | 2692 ± 16^{c} |
| (cP) | | | |
| Trough Viscosity | 1969 ± 12^{a} | 2211 ± 42^{b} | 2356 ± 57^{c} |
| (cP) | | | |
| Breakdown | 196 ± 17^{a} | 367 ± 32^{b} | 335 ± 62^{b} |
| Viscosity (cP) | | | |
| Final Viscosity | 4937 ± 99^{a} | 5044 ± 26^{a} | 5437 ± 123^{b} |
| (cP) | | | |
| Setback | 2967 ± 91^{a} | 2835 ± 62^{ab} | 3080 ± 72^{b} |
| Viscosity (cP) | | | |

TABLE 9
4% sample addition

| Pasting | | | |
|---|---|---|---|
| Parameter | Control | P. ovata | P. turrifera |
| Pasting | 88.95 ± 0.31^{c} | 87.15 ± 0.53^{b} | 69.95 ± 0.33^{a} |
| Temperature (° C.) | | | |
| Peak Viscosity | 2166 ± 6^{a} | 2898 ± 44^{c} | 2688 ± 38^{b} |
| (cP) | | | |
| Trough Viscosity | 1969 ± 12^{a} | 2404 ± 30^{b} | 2432 ± 45^{b} |
| (cP) | | | |
| Breakdown | 196 ± 17^{a} | 565 ± 26^{c} | 266 ± 12^{b} |
| Viscosity (cP) | | | |
| Final Viscosity | 4937 ± 99^{b} | 5525 ± 59^{c} | 4356 ± 66^{a} |
| (cP) | | | |
| Setback | 2967 ± 91^{b} | 3192 ± 44^{c} | 1947 ± 15^{a} |
| Viscosity (cP) | | | |

### Example 8—Effect of Plantago ovata and Plantago turrifera Addition on Syneresis of Rice Flour Pastes

The extent of syneresis was measured on rice flour/Plantago food additive blends at the 4% addition level. Pastes were prepared as per the measurement of standard temperature pasting properties. Triplicate samples of 5 g were stored at 4° C. for 14 days and water separated from the gels was removed every two days and weighed. Syneresis was calculated as the ratio of mass of water removed to the mass of the stored paste.

Syneresis is the process whereby a gel system will spontaneously expel water due to molecule rearrangement during storage. The water affinity of an additive like heteroxylan will reduce the rate at which gel syneresis will occur. Rice flour pastes were used as a control as syneresis is extensive. By adding P. ovata and P. turrifera to the rice flour paste, syneresis was reduced by around 40%, with P. turrifera reducing syneresis significantly more than P. ovata (P<0.01). Additionally, the onset of syneresis was delayed in P. turrifera (4 days of storage vs 2 days in P. ovata). These effects are attributed to the water-holding capacity of the Plantago heteroxylan. The results are shown in FIG. 4.

### Example 9—Influence of P. ovata and P. Turrifera Addition on the Rheological Properties of Rice Flour Dough

Doughs used for rheological tests were prepared in triplicate at breadmaking formulation levels (Table 12) but excluding dried yeast. Viscoelastic properties of baking dough formulations were determined by dynamic oscillatory tests on a Physica MCR 301 Rheometer (Anton Paar, Germany) using 25 mm serrated parallel plate geometry (PP25/P2) with a gap of 2 mm. Excess dough was trimmed and the newly exposed surface was coated in a thin layer of mineral oil to prevent moisture loss. Samples were rested for 500 sec. Oscillatory measurement of the storage (G′) and loss (G″) dynamic moduli was performed at 30° C. (proofing temperature) within a frequency range of 0.1-100 Hz (0.6 to 600 rad/s). The frequency sweep was performed at a constant strain of 0.02%, within the linear viscoelastic region which was determined by amplitude sweep (0.01-10 000% strain) previously (data not shown). The dynamic moduli, G′ and G″, were extracted at 1 Hz and the loss tangent (Tan δ) was calculated as the ratio between G″ and G′.

TABLE 10
Rheological properties

| | | | Rice Flour + |
|---|---|---|---|
| Rheological | | Rice Flour + | P. turrifera |
| property | Rice Flour | P. ovata Flour | Flour |
| Storage | 2610 ± 25^{a} | 7520 ± 407^{b} | 8832 ± 154^{c} |
| Modulus | | | |
| (G′, Pa) | | | |
| Loss Modulus | 375.6 ± 6.4^{a} | 1362.9 ± 47.7^{b} | 1801.9 ± 106.8^{c} |
| G″ (Pa) − 1 Hz | | | |
| Loss Tangent | 0.144 ± 0.004^{a} | 0.181 ± 0.003^{b} | 0.204 ± 0.003^{c} |
| Tan δ − 1 Hz | | | |

Values followed by different letters are highly significantly different (P<0.001)

The storage modulus G′ is a rheological parameter that describes how much energy is stored by a material under deformation and used to reform its original shape. This is generally used to describe strength or elasticity of a material. The addition of hydrocolloids like heteroxylan with a high water affinity and a capacity to form gels will lead to an increase in storage modulus. Rice flour alone has a low storage modulus due to the lack of gluten and viscous polysaccharides. The addition of P. ovata and P. turrifera to a rice flour dough significantly increased the storage modulus with P. turrifera having a significantly higher increase than P. ovata. This shows that P. turrifera heteroxylan forms stronger gels than that of P. ovata.

### Example 10—Dough Proofing Dynamics

Proofing kinetics were studied following Vidaurre-Ruiz et al. (2019) with modifications. Dough was moulded into a cylinder (Ø 22 mm) in the centre of a Petri dish. Cylinders were removed and the doughs were proofed in an incubator at 30° C. for 85 min. Dishes were scanned (C7270i, Canon, Japan) prior to and after rest and every 10 min during proofing. Images were analysed using the measurement function of Photoshop CC 2018 (Adobe, USA). Rest spread (RS) was calculated as the difference in dough circumference at 0 min and 10 min. Proofing changes are presented as the difference between the dough circumference at the time of measurement and after 10 min rest.

Dough proofing dynamics is used to measure kinetic changes in dough size. Rest spread and proofing growth are directly related to the rheological strength of the dough. Rice flour doughs spread at rest and expand during proofing in an uncontrolled way due to poor rheological strength which if occurring in breadmaking leads to uncontrolled growth and collapse. P. ovata reduces rest spread by 46% which will control proofing of the breads but P. turrifera flour reduces rest spread more (75% less than control). Suggesting that the control of growth will be improved and the resultant breads produced will be better.

TABLE 11
Proofing kinetic parameters of gluten-free bread doughs augmented
with the addition of whole seed Plantago flour.

| | | | Rice Flour + |
|---|---|---|---|
| Rheological | | Rice Flour + | P. turrifera |
| property | Rice Flour | P. ovata Flour | Flour |
| Rest Spread | 38.04 ± 1.82^{c} | 17.54 ± 0.61^{b} | 9.6 ± 0.26^{a} |
| (%) | | | |
| Proofing | 43.14 ± 1.5^{b} | 38.02 ± 2.14^{a} | 33.99 ± 1.51^{a} |
| Growth (%) | | | |
| Vₘₐₓ (min⁻¹) | 0.046 ± 0.001^{a} | 0.061 ± 0.002^{a} | 0.103 ± 0.020^{b} |
| X (min) | 38.12 ± 0.52^{b} | 42.94 ± 0.98^{c} | 34.63 ± 0.94^{a} |
| Gompertz | 0.994 | 0.999 | 0.991 |
| Model Fit (R²) | | | |

### Example 11—Breadmaking

The impact of additions of Plantago food additive to baking quality of a rice flour-based gluten-free bread was assessed with baking tests. The effect of Plantago food additive alone was studied by a 4% addition to a rice flour-based formulation (Table 12) without other hydrocolloids or purified starches. Control breads were produced without the addition of Plantago food additive.

TABLE 12
Gluten free bread formulation

| Ingredient | Amount | |
|---|---|---|
| Rice flour | 100 | g |
| Sugar | 5 | g |
| Salt | 2 | g |
| Yeast | 1.5 | g |
| Plantago additive | 4 | g |
| Water | 120 | g |
| Oil | 5 | g |

Doughs and breads were prepared in duplicate and baking and subsequent analyses were performed within one day.

### Baking Procedure

Water and oil were added to the combined dry ingredients and mixed in a multi-speed tilt-head stand mixer (Chef Premier, Kenwood Ltd, UK) with a silicone-edged creaming beater (AT501, Kenwood Ltd) for 7 min at a constant speed. 200 g of dough was transferred to a baking paper-lined multi-size (7.5 cm×7.5 cm×10 cm) anodised cake pan (10034, Silverwood, UK). The pan was sealed with cling film to maintain humidity and doughs proofed at 30° C. for 85 min in an incubator (KB115, Binder, Germany). Proofing of each dough formulation was monitored at the time of baking tests by placing a 10 g sample of dough into a measuring cylinder and proofed at 30° C. for 85 minutes, recording the volume every 10 minutes. The change in dough volume is presented as the difference between the volume at the measurement time and the initial volume.

Loaves were baked at 230° C. for 40 min in a deck oven (Compacta, Tom Chandley, UK), then cooled to room temperature for 1 hr before measurements were taken.

### Bread Qualities

### Bake Loss

Bake loss (water and CO₂ released during baking) was recorded as the difference between dough weight and loaf weight.

$$Bake Loss ( \% ) = \frac{Weight of dough - weight of cooled loaf}{Weight of cooled loaf}$$

### Loaf Volume and Specific Volume

Loaf volume was measured as the displacement of a mass of rapeseeds of known density. Specific volume was calculated as the ratio of the loaf volume to the loaf weight.

### Loaf Imagery and Crumb Structure

Loaves were sliced into 1.25 cm slices (four full slices, two crusts). The central slice surfaces were used for C-Cell imagery. Loaf crumb structure including cell diameter and wall thickness was evaluated using the C-Cell Bread Imaging System (Calibre Control International Ltd., UK) as per the manufacturer's standardised procedure.

### Textural Properties of Bread Crumb

A 30 mm cylindrical punch was used to remove a central representative subsample from each of the four slices for texture analysis. Crumb texture was assessed by texture profile analysis (TPA) using a texture analyser (TA.HDplus, Stable Micro Systems, UK) fitted with a 30 kg load cell and 100 mm aluminium compression platen (P/100, Stable Micro Systems). Pre-test, test, and post-test speeds were 1 mm/s with a target of 65% strain. Hardness, springiness, chewiness and cohesiveness values were calculated by the standard TPA analytical macro.

The TPA ratio was calculated as the ratio between all four TPA parameters.

$$TPA Ratio = ( ( ( Hardness / Cohesiveness ) / Springiness ) / Chewiness )$$

### Moisture Analysis

Moisture analysis was performed by weighing the two central crumb subsamples in an aluminium pan before drying at 105° C. for 24 hours and weighing again. Moisture content was calculated as the difference between fresh weight and dry weight.

TABLE 13
Bread Quality profile

| | | Rice Flour + | Rice Flour + |
|---|---|---|---|
| Quality | | P. ovata | P. turrifera |
| Parameter | Rice Flour | additive | additive |
| Baking loss | 21.58 ± 0.8^{b} | 18.64 ± 0.58^{a} | 17.24 ± 0.12^{a} |
| (%) | | | |
| Specific | 1.479 ± 0.023^{a} | 1.805 ± 0.004^{b} | 1.946 ± 0.006^{c} |
| volume (mL/g) | | | |
| Actual volume | 236.03 ± 5.19^{a} | 294.07 ± 0.83^{b} | 324.68 ± 0.69^{c} |
| (mL) | | | |
| Crumb | 53.77 ± 0.05^{a} | 55.88 ± 0.07^{b} | 55.86 ± 0.07^{b} |
| moisture | | | |
| content (%) | | | |
| Average alveoli | 2.280 ± 0.006^{c} | 1.847 ± 0.029^{b} | 1.709 ± 0.019^{a} |
| diameter (mm) | | | |
| Average | 0.457 ± 0.003^{a} | 0.505 ± 0.005^{b} | 0.469 ± 0.001^{a} |
| intraalveolar | | | |
| wall thickness | | | |
| (mm) | | | |

Baking loss is the amount of dough weight that is lost during cooking. Bake loss is high when gluten-free breads collapse due to loss of moisture or gases. Both P. ovata and P. turrifera flours reduced bake loss from the control as they prevented collapse.

Specific volume is the ratio of volume to weight and is a good measure of loaf ‘airiness’. When breads rise successfully their mass is spread over a larger volume. P. turrifera significantly increased specific volume as the actual volume was high.

Crumb moisture content is a measure of moisture retention. Gluten retains moisture in bread so gluten-free breads are prone to water loss and crumbling. The Plantago flours increased the moisture content by binding water.

Alveoli diameter and wall thickness is a texture parameter. Stronger, more elastic doughs are able to retain small air bubbles during proofing which makes the texture finer and fluffier, which is also due to thin walls between air bubbles. P. turrifera-containing breads have small pores with thin walls which may contribute to the improved texture.

TABLE 14
Bread Texture Profile Analysis

| | | Rice Flour + | Rice Flour + |
|---|---|---|---|
| Quality | | P. ovata | P. turrifera |
| Parameter | Rice Flour | additive | additive |
| Hardness (g) | 2579 ± 121^{a} | 3157 ± 62^{ab} | 2924 ± 135^{b} |
| Cohesiveness | 0.875 ± 0.009^{a} | 0.915 ± 0.005^{b} | 0.945 ± 0.004^{c} |
| Springiness | 0.329 ± 0.006^{a} | 0.525 ± 0.004^{b} | 0.614 ± 0.002^{c} |
| Chewiness (g) | 1193 ± 19^{a} | 1984 ± 35^{b} | 2138 ± 12^{c} |
| TPA Ratio | 7.51 | 3.31 | 2.36 |

Hardness in bread is perceived as staleness or stodginess. It is ideal to have reduced hardness in gluten-free breads but many gelling hydrocolloids increase hardness by through gel formation and binding of water. While it did not improve the hardness, P. turrifera did not significantly increase it.

Cohesiveness, springiness and chewiness are all positive textural traits for bread as it relates to the robustness of the material during eating. Without gluten, gluten-free breads crumble and disintegrate which is sensorially unpleasant. P. turrifera improved all three traits.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

It is to be noted that where a range of values is expressed, it will be clearly understood that this range encompasses the upper and lower limits of the range, and all numerical values or sub-ranges in between these limits as if each numerical value and sub-range is explicitly recited. The statement “about X % to Y %” has the same meaning as “about X % to about Y %,” unless indicated otherwise.

The term “about” as used in the specification means approximately or nearly and in the context of a numerical value or range set forth herein is meant to encompass variations of +/−10% or less, +/−5% or less, +/−1% or less, or +/−0.1% or less of and from the numerical value or range recited or claimed.

It is also to be noted that, as used herein, the singular forms “a”, “an” and “the” include plural aspects unless the context already dictates otherwise.

The subject headings used herein are included only for the ease of reference of the reader and should not be used to limit the subject matter found throughout the disclosure or the claims. The subject headings should not be used in construing the scope of the claims or the claim limitations.

The description provided herein is in relation to several embodiments which may share common characteristics and features. It is to be understood that one or more features of one embodiment may be combinable with one or more features of the other embodiments. In addition, a single feature or combination of features of the embodiments may constitute additional embodiments.

All methods described herein can be performed in any suitable order unless indicated otherwise herein or clearly contradicted by context. The use of any and all examples, or exemplary language (e.g., “such as”) provided herein, is intended merely to better illuminate the example embodiments and does not pose a limitation on the scope of the claimed invention unless otherwise claimed. No language in the specification should be construed as indicating any non-claimed element as essential.

It will be apparent to the person skilled in the art that while the invention has been described in some detail for the purposes of clarity and understanding, various modifications and alterations to the embodiments and methods described herein may be made without departing from the scope of the inventive concept disclosed in this specification.

Those skilled in the art will appreciate that the invention described herein is susceptible to variations and modifications other than those specifically described. It is to be understood that the invention includes all such variations and modifications. The invention also includes all of the steps, features, compositions and compounds referred to, or indicated in this specification, individually or collectively, and any and all combinations of any two or more of the steps or features.

Future patent applications may be filed in Australia or overseas on the basis of the present application, for example by claiming priority from the present application, by claiming a divisional status and/or by claiming a continuation status. It is to be understood that the following provisional claims are provided by way of example only and are not intended to limit the scope of what may be claimed in any such future application. Furthermore, the claims should not be considered to limit the understanding of (or exclude other understandings of) the invention inherent in the present disclosure. Features may be added to or omitted from the provisional claims at a later date, so as to further define the invention.

1. A food additive composition comprising flour or meal made from seeds of a plant of the genus Plantago, wherein the ω-3 to 0-6 fatty acid molar ratio of the seeds is at least 2.0:1.

2. A food additive composition according to claim 1 wherein the ω-3 to ω-6 fatty acid molar ratio of the seeds is at least 3.0:1.

3. (canceled)

4. A food additive composition according to claim 1 wherein the ω-3 to ω-6 fatty acid molar ratio of the seeds is at least 4.5:1.

5. A food additive composition according to claim 1 wherein the ω-3 to ω-6 fatty acid molar ratio of the composition is at least 4.0:1.

6. A food additive composition according to claim 1 wherein the ω-3 to ω-6 fatty acid molar ratio of the composition is at least 4.5:1.

7. A food additive composition according to claim 1 wherein the plant of the genus Plantago is a species that contains a sequence having at least 95% sequence identity to sequence ID No 1.

8. (canceled)

9. A food additive composition according to claim 1 wherein the plant of the genus Plantago is a species that contains a sequence having at least 99% sequence identity to sequence ID No 1.

10. A food additive composition according to claim 1 wherein the plant of the genus Plantago is Plantago turrifera or a variant or mutant thereof.

11. (canceled)

12. A food additive composition according to claim 1 wherein the composition contains alpha linoleic acid in an amount of at least 5 wt % based on the total weight of the composition.

13. A food additive composition according to claim 1 wherein the composition contains alpha linoleic acid in an amount of at least 6 wt % based on the total weight of the composition.

14. A food additive composition according to claim 1 wherein composition has a weight average particle size of from 1 to 1000 micron.

15. A baking composition comprising (1) a food additive composition according to claim 1 and (2) flour.

16. A baking composition according to claim 15 wherein the composition comprises from 1 wt % to 6 wt % of the food additive composition and from 94 wt % to 99 wt % flour or wherein the composition comprises 3 wt % to 5 wt % of the food additive composition and 95 wt % to 97 wt % of the flour.

17. (canceled)

18. A baking composition according to claim 16 wherein the flour is a gluten free flour.

19. A baking composition according to claim 18 wherein the gluten free flour is selected from the group consisting of almond flour, brown rice flour, buckwheat flour, chickpea flour, corn flour, rice flour, sorghum flour, tapioca flour, teff flour or a combination thereof.

20. A baking composition according to claim 15 wherein the flour is rice flour.

21. A method of making a baked product, the method comprising the steps of:
(a) forming a dough comprising (1) a food additive composition according to claim 1 and (2) a flour; and
(b) baking the dough to form a baked product.

22. (canceled)

23. A method according to claim 21 wherein the flour is a gluten free flour.

24. A method according to claim 23 wherein the gluten free flour is selected from the group consisting of almond flour, brown rice flour, buckwheat flour, chickpea flour, corn flour, rice flour, sorghum flour, tapioca flour, teff flour or a combination thereof.

25-26. (canceled)

27. A method of making a food additive composition the method comprising providing seeds of a plant of the genus Plantago, wherein the seeds have ω-3 to ω-6 fatty acid molar ratio of at least 2.0:1, and grinding the seeds to produce a flour or meal.
